(19) United States
(12) Patent Application Publication
(10) Pub. No.: US 2005/0283924 A1
(43) Pub. Date: Dec. 29, 2005

(54) Color compositions and methods of using the same

(75) Inventor: Richard Kaiser, Allentown, PA (US)

Correspondence Address: AKERMAN SENTERFITT, P.O. BOX 3188, WEST PALM BEACH, FL, 33402-3188, US

(73) Assignees: Spectrum Associates LLC, Boca Raton, FL (US); Longoria Design LLC, Miami, FL (US); Kennedy/Matsumoto Design LLC, Lantana, FL (US)

(21) Appl. No.: 11/165,108

(22) Filed: Jun. 23, 2005

Related U.S. Application Data
(63) Continuation-in-part of application No. 10/877,611, filed on Jun. 25, 2004.

(51) Int. Cl. A61K 8/00

(52) U.S. Cl. 8/405

(57) ABSTRACT

A temporary hair color composition and a method of using this color composition. The temporary hair color composition is in a form suitable for application to hair and includes water, a co-solvent, a gelling agent selected from a soap gelling agent and a gellan gum gelling agent, and a coloring agent. The color composition results in a that may by applied using pressure and which does not drip. As such, the color composition may be easily applied. The color compositions may be used such that the same color results even though the color of the underlying substrate is substantially different.

### FIELD OF THE INVENTION

This invention is directed generally to color compositions, and more particularly to color compositions and methods for applying these color compositions to different portions of an individual's anatomy.

### BACKGROUND OF THE INVENTION

Many individuals color one or more portions of the anatomy for a variety of different reasons. For example, many individuals may color certain portions of their skin, such as their lips, face and hands. Applying color to the lips and/or face is often done by many women as part of their daily routine. However, on various occasions, men may use color as well, such as for Halloween or costume parties. In many instances, these individuals may wish to try the coloring for a shorter period of time, with the ability to remove the coloring if they do not like their new look and/or once the special occasion is finished.

For the same or similar reasons, some individuals color their hair. Hair coloring may be undertaken to change or cover the color of hair for many different reasons. For instance, hair is often colored to cover hair that has turned gray, to lighten or change the shade of hair, to highlight or lowlight hair, to try something new, or for a special occasion, again, such as dressing up for Halloween. The hair coloring procedures used to generate these results are often markedly different and use different coloring materials. The hair coloring materials may be dyes that may or may not be easily washed from the colored hair with conventional shampoo and water, or other materials. Again, in many instances, these individuals may wish to try the coloring for a shorter period of time, with the ability to remove the coloring if they do not like their new look or once the special occasion is finished.

The application of cosmetic and personal care products to the skin almost always involves the formation of a film on the skin. For example, application of a foundation to the face involves formation of a colored film on the skin. The same is true of products such as lipstick, eye shadow, blush, and nail enamel. The product is applied to the appropriate keratinous surface and allowed to dry. A film is formed that adheres to the skin for the appropriate period of time before being removed by chemical processes, or washed off with soap and/or water.

Accordingly, film forming polymers are widely used in the cosmetic industry. The ability to form films on skin, lips, or hair is one of the main means of providing a cosmetic benefit. For example, materials such as film forming cationic polymers are found in hair conditioning agents. They are capable of depositing on the hair to form a film or coating which provides benefits varying from shine, to softness, manageability, and similar characteristics. Similarly, various film forming polymers may be found in color cosmetics such as lipsticks. These polymers may affect lipstick wear and adhesion, in addition to serving as a film to hold the pigments in place on the lips. Also, nail enamel compositions usually contain, as the main component, some type of film forming polymer. Such compositions are typically wholly liquid or comprise liquids which contain solid particulates in suspension. They are applied as liquids and, after drying, the film formed on the nail usually lasts for at least several days, although a key feature for such cosmetic applications is their removability. It is thus beneficial to provide compositions which are “semi-permanent,” meaning that they demonstrate high durability on the coated substrate, but yet may be easily removed at will by the user using mechanical methods such as wiping or absorbing into a porous medium such as a tissue, as with lipsticks; or by chemical methods, for example, the removal of a nail enamel using an organic solvent such as ethyl acetate or acetone. In general, the types of polymeric materials used in cosmetics have a significant impact on providing compositions with improved properties. Thus, cosmetics companies are always searching for new and different polymers to provide properties which improve cosmetic performance.

Most film forming cosmetic and personal care products contain a polymeric material as the primary film former. The term “film former” as used herein refers to a material that, upon drying, produces a continuous film on keratinous substrates such as skin, hair, or nails. The term “film forming polymer” as used herein refers to a film former that is in the polymeric form. A variety of polymers have film forming properties: they may be natural polymers, synthetic polymers, or polymers that have both natural and synthetic portions. While the polymers available today have a myriad of properties, most cosmetics and personal care products contain other ingredients to further modify the properties of the composition and improve aesthetics.

One of these additional ingredients is a colorant. Many make-up compositions, such as free or compact powders, foundations, face powders, eye shadows, lipsticks, concealers, blushers, mascaras, eyeliners, lip pencils or eye pencils or make-up products for the body, include a suitable vehicle and coloring agents of various natures, intended to give these compositions a certain color, before and/or after applying them to the skin, the lips and/or hair.

These coloring agents may be lakes, inorganic or organic pigments and/or pearlescent pigments, or, alternatively, dyes. However, many of these agents may present problems. Inorganic pigments have the advantage of being relatively stable, but have the drawback of giving rather dull, pale colors. Organic lakes have the advantage of giving the compositions lively colors, but are mostly unstable with respect to light, temperature or pH. As for pearlescent pigments, they allow varied, but never intense, colors to be obtained, with iridescent effects, but these are usually fairly weak.

Accordingly, it would be beneficial to provide a color composition that may be used to color various portions of a person's anatomy, such as hair, lips, face, hands, or the like. It would also be beneficial to provide a color composition that is semi-permanent such that it may be applied and removed after a short period of time. It would also be beneficial to provide a color composition that provides a more visible color, regardless of the color of the substrate to which it was applied.

### SUMMARY OF THE INVENTION

The present invention provides a color composition and a method of applying this composition to any part of a user's anatomy, and more particularly, the hair. The color composition results in a material that may by applied using slight pressure, and which does not drip. As such, the color composition may be easily applied. Various embodiments of the color composition result in differing colors and may even be used for metallic colors. In some embodiments, the color compositions may be used such that the same color results even though the color of the underlying substrate is substantially different, such as blonde hair versus brunette hair.

In particular, in one aspect, the invention relates to a method for temporarily modifying the color of hair, which includes the step of providing a composition comprising water, a co-solvent, a gelling agent selected from a soap gelling agent and a gellan gum gelling agent, and a coloring agent. The composition is then applied to the hair. The composition removably adheres to the hair, and washes off with the application of a cleaning agent to the hair.

Another aspect of the invention relates to the temporary hair color composition, which can include water, a co-solvent, a gelling agent selected from a soap gelling agent and a gellan gum gelling agent, and a coloring agent. The temporary hair color composition is in a form suitable for application to hair.

A further aspect of the invention relates to a kit for temporarily modifying the color of hair. The kit includes a package, and a coloring composition provided in the package. The coloring composition includes water, a co-solvent, a gelling agent selected from a soap gelling agent and a gellan gum gelling agent, and a coloring agent. Instructions are provided on or in the package instructing a user to removably adhere the coloring composition to the user's hair by applying the coloring composition to the user's hair.

Yet a further aspect of the invention relates to use of any of the hair coloring compositions described in the present specification for temporarily modifying hair.

In one embodiment, the co-solvent may be a glycol, and the gelling agent may be a soap gelling agent. The glycol may be propylene glycol and the soap gelling agent may be sodium stearate in one embodiment.

The composition can include from about 20 to about 35% by weight of water, from about 20 to about 35% by weight of a co-solvent, from about 10 to about 30% by weight of a soap gelling agent, and from about 15 to about 30% by weight of a coloring agent. The composition can also include from about 1 to about 5% by weight of a silicone polymer.

In an alternative embodiment, the composition can include from about 27 to about 32% by weight of water, from about 26 to about 31% by weight of the co-solvent, and from about 12 to about 15% by weight of soap gelling agent. The co-solvent in this arrangement may be a glycol, and the soap gelling agent may be a stearate salt. The glycol may be propylene glycol, and the stearate salt may be sodium stearate.

In another embodiment, the co-solvent may be a glycol, and the gelling agent may be a gellan gum gelling agent. The gellan gum gelling agent can have low levels of acyl content. In this embodiment, the composition can include from about 20 to about 65% by weight of water, from about 10 to about 35% by weight of a co-solvent, and from about 0.1 to about 2% by weight of a low level acyl content gellan gum. In a preferred arrangement, the composition can include from about 50 to about 65% by weight of water, from about 10 to about 20% by weight of a co-solvent, and from about 0.5 to about 1.5% by weight of a low level acyl content gellan gum.

These and other uses will become apparent upon review of the detailed description below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 is a depiction of the formation of liquid crystals used in a base according to one embodiment of the present invention.

FIG. 2 is a diagram demonstrating possible mixing ratios of three components according to one embodiment of the present invention.

### DETAILED DESCRIPTION OF THE INVENTION

The present invention is more particularly described in the following description and examples that are intended to be illustrative only since numerous modifications and variations therein will be apparent to those skilled in the art. As used in the specification and in the claims, the singular form “a,” “an,” and “the” may include plural referents unless the context clearly dictates otherwise. Also, as used in the specification and in the claims, the term “comprising” may include the meanings “including”, “consisting of” and “consisting essentially of.”

The color composition of the present invention is contemplated to be used on one or more parts of an individual's anatomy, such as by using as a cosmetic. In a preferred embodiment, the color compositions are applied to hair to provide a hair dye that is removable by physical means, such as shampooing. As such, it is beneficial for the composition to include materials that are safe, nontoxic, and/or capable of being approved as cosmetic grade. In addition, as the color compositions may be applied for cosmetic purposes, it may be beneficial for the compositions to suspend color, dry quickly, stick to the substrate, and/or wash off with the application of soap or another cleaning agent. Also, the firmness of the compositions may be varied, depending on the selected characteristics that may be beneficial for application to the particular body part.

Accordingly, in one aspect of the present invention, the color composition utilizes liquid crystal technology as compositions made using this technology are safe, are suitable for application to skin and hair, and/or may be made into a wide range of hardness. The liquid crystal technology is used to produce the base to the color composition. The base, in conjunction with a coloring agent and optional ingredients, forms the color composition of the present invention.

The base of the coloring compositions is preferably a soap gelling agent. A soap gelling agent, such as sodium stearate, has a structure wherein one end of the molecule is hydrophilic and the other end is oleophilic. As such, when the soap gelling agent is dissolved in water and a miscible solvent, the soap gelling agent molecule is capable of orienting one end in each solvent. Due to the optimum packing order of, for example, the sodium stearate molecules, tubes or liquid crystals may be formed. These tubes can actually partition the water and the co-solvent. The soap gelling agents hereof can be salts of fatty acid containing from about 12 to about 40 carbon atoms (C₁₂-C₄₀), preferably salts of C₁₂-C₂₂ fatty acids, more preferably C₁₄-C₂₀, and most preferably C₁₆-C₂₀. Suitable salt forming cations for use in these gelling agents include metal salts such as alkali metals, e.g. sodium and potassium, alkaline earth metals, e.g., magnesium, and aluminum. Preferred are sodium and potassium salts.

In addition to the soap gelling agent and water, the coloring compositions can use a co-solvent. The co-solvent can be any co-solvent capable of forming a coloring composition with the soap gelling agent (e.g., sodium stearate) and water. Thus, the co-solvent can be a glycol, for example, propylene glycol. Other glycols or polyhydric alcohols can also be used, such as dipropylene glycol, ethylene glycol, diethylene glycol, butylene glycol, hexylene glycol, glycerol, glycerin, or any glycol mentioned in U.S. Pat. No. 6,299,891, which is hereby incorporated by reference. When sodium stearate is dissolved in water and propylene glycol, a liquid crystal forms, with the oleophilic end of the sodium stearate oriented toward the propylene glycol, and water oriented at the hydrophilic end of the sodium stearate. As the material cools, the crystals form, with the length of the crystal being determined by the rate of cooling. The faster the cooling rate, the shorter the crystal and the shorter the cooling rate, the longer the crystal.

The length of the crystal has an effect on the mechanical properties of the resulting composition. Longer crystals are generally more flexible and, as a result, are generally weaker than shorter crystals. As such, shorter crystals are comparably stronger. For example, a composition having a mixture of 33% short crystals and 66% liquid may be as strong as a composition that is 100% hard wax.

The overall physical properties of hair coloring compositions that include these preferred materials is determined by the ratio of the soap gelling agent, water, and co-solvent. If sodium stearate is selected to be the soap gelling agent, then the ratio of the water to co-solvent to sodium stearate determines the concentration of the crystals and the continuous phase.

Preferably, the water and glycol are present in substantially equal amounts, wherein the soap gelling agent is present in a lesser amount. The cosmetic compositions having such relative amounts of water, co-solvent (e.g. propylene glycol) and soap gelling agent (e.g. sodium stearate) provide a consistency such that it can be easily applied to, for example, hair. Thus, the amount of soap gelling agent (e.g., sodium stearate) in the coloring composition can be from about 15 to about 50% less than the amount of the water and the co-solvent. As such, the hair coloring composition includes from about 20 to about 35% by weight of the total coloring composition of water and the co-solvent, and from about 10 to about 30% by weight of sodium stearate. More preferably, the hair coloring composition includes from about 26 to about 32% by weight of the total coloring composition of water and the co-solvent, and from about 12 to about 15% by weight of sodium stearate.

In an alternative embodiment, the composition comprises from about 1.5 or 2 to about 3 times more water than co-solvent (e.g. propylene glycol). The color composition according to this embodiment includes from about 30 to about 50% by weight of water, and from about 15% to about 30% by weight of a co-solvent. As compared to the preferred embodiment in which the amount of water and co-solvent (e.g. propylene glycol is about equal, the color composition of this embodiment have a harder consistency, and are thus not as conducive to easy application to a surface such as hair. It has been found, however, that compositions according to this alternative embodiment are not as prone to leaching water in high temperature and humidity environs.

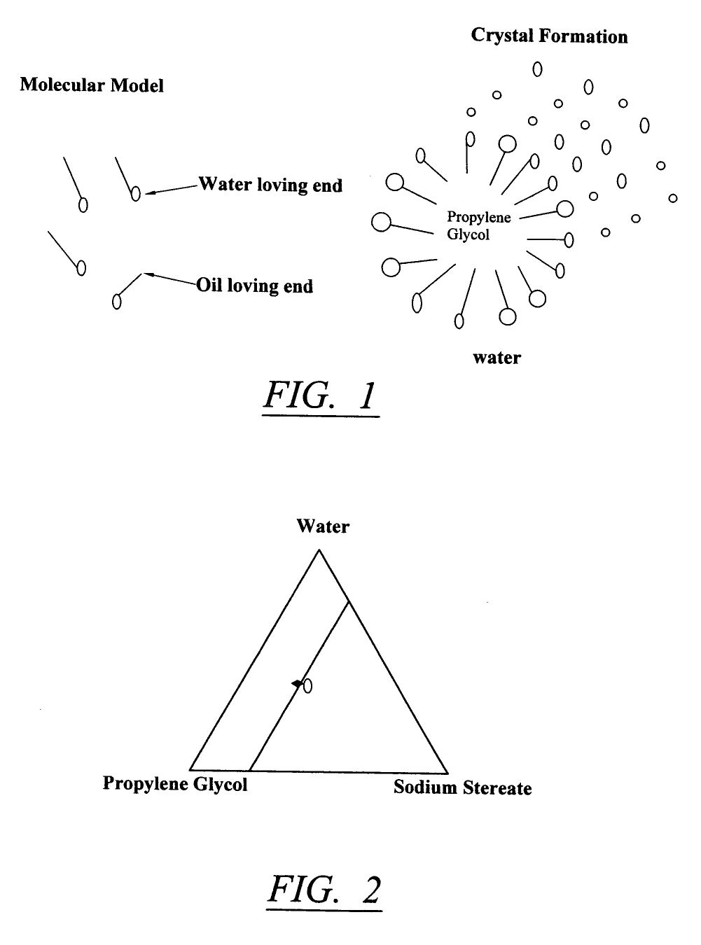

FIG. 1 provides a depiction of the formation of liquid crystals in an embodiment wherein the co-solvent is propylene glycol and the soap gelling agent is sodium stearate. As seen, the sodium stearate has a hydrophilic end (as shown with a water molecule attached thereto) and an oleophilic end. Crystals form when the oleophilic ends of the sodium stearate align towards the propylene glycol.

The overall physical properties of the color compositions of the present invention may be determined by the ratio of the soap gelling agent, water, and co-solvent. In an embodiment wherein sodium stearate is the soap gelling agent, the ratio of the water to co-solvent to sodium stearate determines the concentration of the crystals and the continuous phase. FIG. 2 provides a diagram demonstrating the possible mixing ratios of these three components in an embodiment wherein the co-solvent is propylene glycol.

As shown in FIG. 2, the oval in the center represents the center of the diagram wherein each component is present in equal amounts. Formulations having more water and/or sodium stearate tended to be too hard or too mushy. Formulations having an excess amount of propylene glycol in relation to the water also tended to be too mushy. It was discovered that compositions in the area represented by the diamond offered the most beneficial characteristics, although formulations around the diamond may also be used, depending on the selected characteristics of the final color composition and/or the co-solvent used. Nevertheless, in those embodiments represented by the diamond, the water and propylene glycol are present in substantially equal amounts, wherein the sodium stearate is present in a lesser amount. In select embodiments, the amount of sodium stearate in the color composition is from about 15 to about 50% less than the amount of the water and the co-solvent. As such, in one embodiment, the present invention includes from about 20 to about 35% by weight of the total color composition of water and the co-solvent, and from about 10 to about 30% by weight of sodium stearate. In another embodiment, the present invention includes from about 26 to about 32% by weight of the total color composition of water and the co-solvent, and from about 12 to about 15% by weight of sodium stearate.

In an alternative embodiment, the soap gelling agent is replaced with a polysaccharide gelling agent, preferably a heteropolysaccharide gelling agent such as a gellan gum having repeating glucose, rhamnose and glucuronic acid units. Gellan solutions are a heteropolysaccharide obtained via the fermentation of the bacteria Sphingomonas elodea ATCC 31461 (previously called Pseudomonas elodea). Gellan gums, also commonly known as gellan gum fluid gels, can be prepared by adding an electrolyte (e.g., a salt, an acid or an anionic surfactant) to a hot gellan solution and then cooling. Gellan gum is available from Kelco Division of Monsanto Co., San Diego, Calif. under various names, including Kelcogel™, Kelcogel PC™, and Kelcogel F™ and also include those described in U.S. Pat. Nos. 5,654,027, 4,326,052 and 4,326,053, each which are hereby incorporated by reference.

Gellan gums are available in two types—high and low acyl content. The high acyl form produces soft, elastic, non-brittle gels and has the structure shown below: 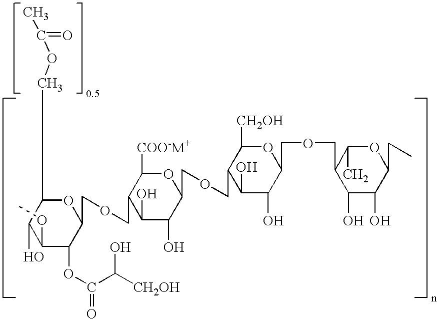
The low acyl form produces firm, non-elastic, brittle gels and has the structure shown below: 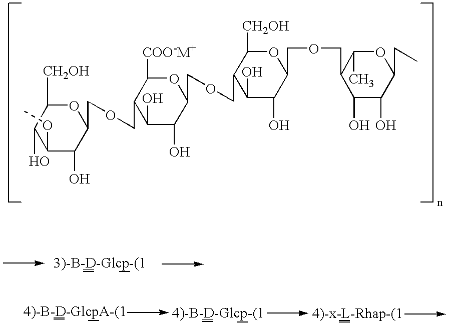
A preferred polysaccharide gelling agent is Kelcogel F™.

Gellan gum can be extremely effective at low use levels and forms solid gels at low concentrations, especially as compared to the amount sodium stearate otherwise employed in the coloring compositions. In one embodiment, the coloring composition comprises from about 20 to about 65% water, from about 10 to about 35% of co-solvent, and from about 0.1 to about 2% of gellan gum. In one embodiment, the color compositions comprises from about 50 to about 60% water, from about 10 to about 20% co-solvent, and from about 0.5 to about 1.5% gellan gelling agent. In a preferred embodiment, the co-solvent is propylene glycol and the gellan gum is Kelcogel F™.

The base coloring composition optionally includes a component to improve the spreadability of the coloring composition, thereby making the composition easier to apply to the hair. For example, this component can be a silicone polymer. One example of a silicone polymer that may be used is Gafquat H—Si, which is manufactured by International Specialty Products (ISP, Wayne, N.J.), though other sources for functionally equivalent silicone polymers exist. The amount of the silicone polymer added can range from about 1% to about 5% by weight of the total coloring composition. A preferred amount of silicone polymer is about 2% by weight of the total coloring composition. A coloring agent is added to the base. One of the problems with conventional coloring agents is that not all colors appear the same when applied to substrates having different colors. For example, a blue color applied to blonde hair may appear different than the same blue color applied to black hair as the “blueness” is much less noticeable on the darker substrate. It is preferred that the coloring agent in the color composition 12 result in a coloring composition that appears substantially the same, regardless of the color of the substrate to which it is applied.

Accordingly, the coloring agent preferably includes titanium dioxide and a colorant. The titanium dioxide is used to effectively “hide” or cover up the color of the underlying substrate such that the color of the colorant may be seen regardless of the color of the underlying substrate. As such, the coloring composition 12 can be applied to all types and colors of hair. The amount of titanium dioxide can range from about 5 to about 20% by weight of the total coloring composition. More preferably, the amount of titanium dioxide is from about 7 to about 15% by weight of the total coloring composition. Other materials besides titanium dioxide can be used including, but not limited to, barium sulfate, aluminum silicates, calcium carbonates, and combinations thereof or, more generally, appropriate metal salts that permit the colorants in the composition to show true when applied to hair of varying color.

When titanium dioxide or a related material is used as part of the coloring agent, the coloring agent includes a colorant. The colorant is the component that actually provides the color that is seen. The amount of colorant to be used will vary depending on the amount of titanium dioxide, the color of the colorant, and/or the selected degree of color for the finished coloring composition. Illustratively, the total amount of titanium dioxide and colorant is from about 15 to about 30% by weight of the total coloring composition. More preferably, the total amount of titanium dioxide and colorant is from about 20 to about 25% by weight of the total coloring composition. Accordingly, the amount of colorant can be from about 5 to about 20% by weight of the total coloring composition, and more preferably from about 7 to about 15% by weight of the total coloring composition. Examples of colorants useful in the present invention include, but are not limited to, Sandsperse Blue WF253, Napthol Red 2R Dispersion, and Optiflow Yellow 74. Other colorants that can be used include those listed by the Food and Drug Administration under Color Additives Approved for Use in Cosmetics (Part 73, Subpart C and Part 74, Subpart C) as currently listed and/or as amended in the future. This publication is hereby incorporated by reference.

Optionally, the coloring composition can-include a metallic coloring agent to produce a metallic color. When a metallic coloring agent is used, no titanium dioxide is required. One metallic coloring agent is a mica-based metallic. As no titanium dioxide is necessary, additional amounts of the metallic coloring agent can be used in the final coloring composition. Accordingly, the amount of the metallic coloring agent van range from about 15 to about 25% by weight of the total coloring composition, and more preferably is about 20% by weight of the total coloring composition. Examples of metallic coloring agents useful in the present invention include, but are not limited to, DynaColor 98392B15A BlueGreen, Allair Type 303 Gold, and DynaColor RB639XV19A available from Dynacolor Inc. Other metallic coloring agents that can be used include those metallic coloring agents listed by the Food and Drug Administration under Color Additives Approved for Use in Cosmetics (Part 73, Subpart C and Part 74, Subpart C) as currently listed and/or as amended in the future.

The color compositions may also include a dye-binder, which will bind the colorant or dye and prevent them from leaching out of the color composition upon storage or staining the hands upon handling the color composition. An color compositions of the present invention to form phosphomolybdic acid. Other dye-binders which can be used in the present invention are set forth in Industrial Organic Pigments: Production, Properties, Applications, 3rd Edition, by Willy Herbst et al, which is hereby incorporated by reference in its entirety.

In addition to the base and the coloring agent, the coloring compositions of the present can include other optional components for a variety of purposes. For example, a dispersant for preventing agglomeration between the pigment particles in the coloring composition. One example of a dispersant that can be used in the present invention is Tamol 731, a sodium salt of a carboxylated polyelectrolyte available from Rohm & Haas (Philadelphia, Pa.). Another example of a dispersant is Thorcowet G-26, a glycerin derivative available from the Thornley Company (Wilmington, Del.), which has been found to be particularly effective when used with oleophilic pigments. The dispersant can be added in an amount of from about 0 to about 5% by weight of the total coloring composition. Other dispersants can be used, including those manufactured by, e.g., Olin, Rohm & Haas, Stefan. Examples of dispersants include, without limitation, soluble salts of low molecular weight homopolymers or copolymers of polyacrylic acids, partially hydrolyzed polyacrylamides, maleic anhydride copolymers, and polyaspartic acid.

The coloring composition also can include a bactericide to prevent growth of bacteria in the product prior to use. One example of a suitable bactericide is Germall®, a member of the substituted imidazolidinyl urea family of compounds available from ISP. The bactericide may be added in an amount of from about 0 to about 1% by weight of the total coloring composition. Other commercially available bactericides, such as quaternary ammonium compounds may be used. In addition, other bactericides and fungicides may also be used.

The coloring composition can include a defoaming agent to help control foaming during production of the compositions of the present invention. One example of a suitable defoaming agent is Foamstar™ A-12, a polymer available from Cognis (Cincinnati, Ohio). Another example of a defoaming agent is Chemburst A-20FG, an emulsion available from Chemsil Silicones (Chatsworth, Calif.). Chemburst A-20FG has been found to be particularly useful when used with oleophilic pigments. The defoaming agent can be added in an amount of from about 0 to about 3% by weight of the total coloring composition.

The coloring composition 12 can be made by mixing the water and co-solvent together, then heating to a temperature above which the soap gelling agent melts, generally above about 60° C., to form the base. Then, the coloring agent and any optional compounds as noted above can be added and the mixture heated to a temperature of from about 90 to about 100° C. to thoroughly mix all of the components. Then, the mixture is preferably cooled gradually to form the liquid crystals and the coloring compositions. The rate of cooling is preferably controlled to control the final strength of the coloring composition, wherein a faster rate of cooling results in a softer composition and a slower rate of cooling results in a firmer composition.

The compositions of the present invention may be used to color a variety of different surfaces including, but not limited to, various parts of an individual's anatomy. These various anatomy parts of an individual may include, but are not limited to, the hair, skin, lips or other surface of an individual. The compositions may be applied in any known manner, such as forming the composition into a stick and applying to the hair, lips, and/or face. In another embodiment, the compositions may be formed into a compact or any other suitable shape and applied using an applicator. As used herein, an “applicator” is any item that may be used to apply a semi-solid or substantially solid material to a surface including, but not limited to, fingers, a brush, a pad, cotton, a sponge, a fabric, and the like.

The foregoing is provided for purposes of illustrating, explaining, and describing embodiments of this invention. Modifications and adaptations to these embodiments will be apparent to those skilled in the art and may be made without departing from the scope or spirit of this invention.

### EXAMPLES

### Example 1

An example of a blue composition made according to various aspects of the present invention. In a mixing vessel, the following components were added:

| | | |
|---|---|---|
| Deionized water | 28 | parts |
| Propylene Glycol | 27.8 | parts |
| Tamol 731 | 4 | parts |
| Gafquat H—Si | 2 | parts |
| Foamstar A-12 | 1 | part |
| Germal | 0.2 | parts |

The mixing vessel was heated to 80° C. and 15 parts titanium dioxide were added. The composition was mixed until all lumps were dispersed and the fluid appeared to be free of grit. Then, the colorant was added, 7 parts of Sandsperse Blue WF253 and the composition was heated until 80° C. Then 15 parts sodium stearate was added while ensuring the temperature did not drop below 60° C. The vessel was heated to 101° C. and held for 5 minutes before slowly cooling to form the final composition.

### Example 2

An example of a red composition made according to various aspects of the present invention. In a mixing vessel, the following components were added:

| | | |
|---|---|---|
| Deionized water | 28.8 | parts |
| Propylene Glycol | 28 | parts |
| Tamol 731 | 4 | parts |
| Gafquat H—Si | 2 | parts |
| Germal | 0.2 | parts |

The mixing vessel was heated to 80° C. and 7 parts titanium dioxide were added. The composition was mixed until all lumps were dispersed and the fluid appeared to be free of grit. Then, the colorant was added, 15 parts of Napthol Red 2R Dispersion and the composition was heated until 80° C. Then 15 parts sodium stearate was added while ensuring the temperature did not drop below 60° C. The vessel was heated to 101° C. and held for 5 minutes before slowly cooling to form the final composition.

### Example 3

An example of a yellow composition made according to various aspects of the present invention. In a mixing vessel, the following components were added:

| | | |
|---|---|---|
| Deionized water | 27.5 | parts |
| Propylene Glycol | 26.3 | parts |
| Tamol 731 | 4 | parts |
| Gafquat H—Si | 2 | parts |
| Germal | 0.2 | parts |

The mixing vessel was heated to 80° C. and 15 parts titanium dioxide were added. The composition was mixed until all lumps were dispersed and the fluid appeared to be free of grit. Then, the colorant was added, 10 parts of Optiflo Yellow and the composition was heated until 80° C. Then 15 parts sodium stearate was added while ensuring the temperature did not drop below 60° C. The vessel was heated to 101° C. and held for 5 minutes before slowly cooling to form the final composition.

### Example 4

An example of an electric blue metallic composition made according to various aspects of the present invention. In a mixing vessel, the following components were added:

| | | |
|---|---|---|
| Deionized water | 29.8 | parts |
| Propylene Glycol | 28 | parts |
| Tamol 731 | 4 | parts |
| Gafquat H—Si | 2 | parts |
| Foamstar A-12 | 1 | part |
| Germal | 0.2 | parts |

The mixing vessel was heated to 80° C. and the coloring agent, 20 parts Dyna Color 98392B15A BlueGreen, was added. The composition was mixed until all lumps were dispersed and the fluid appeared to be free of grit. Then 15 parts sodium stearate was added while ensuring the temperature did not drop below 60° C. The vessel was heated to 101° C. and held for 5 minutes before slowly cooling to form the final composition.

### Example 5

An example of a gold metallic composition made according to various aspects of the present invention. In a mixing vessel, the following components were added:

| | | |
|---|---|---|
| Deionized water | 29.8 | parts |
| Propylene Glycol | 28 | parts |
| Tamol 731 | 4 | parts |
| Gafquat H—Si | 2 | parts |
| Foamstar A-12 | 1 | part |
| Germal | 0.2 | parts |

The mixing vessel was heated to 80° C. and the coloring agent, 20 parts Allair Type 303 Gold, was added. The composition was mixed until all lumps were dispersed and the fluid appeared to be free of grit. Then 15 parts sodium stearate was added while ensuring the temperature did not drop below 60° C. The vessel was heated to 101° C. and held for 5 minutes before slowly cooling to form the final composition.

### Example 6

An example of a raspberry metallic composition made according to various aspects of the present invention. In a mixing vessel, the following components were added:

| | | |
|---|---|---|
| Deionized water | 29.8 | parts |
| Propylene Glycol | 28 | parts |
| Tamol 731 | 4 | parts |
| Gafquat H—Si | 2 | parts |
| Foamstar A-12 | 1 | part |
| Germal | 0.2 | parts |

The mixing vessel was heated to 80° C. and the coloring agent, 20 parts Dyna Color RB639XV19A, was added. The composition was mixed until all lumps were dispersed and the fluid appeared to be free of grit. Then 15 parts sodium stearate was added while ensuring the temperature did not drop below 60° C. The vessel was heated to 101° C. and held for 5 minutes before slowly cooling to form the final composition.

### Examples 7-14

Using the process set forth in Examples 1-6, eight additional formulations were prepared. The listing of the ingredients for these formulations is set forth in Table 1.

TABLE 1
HAIR PAINT INGREDIENTS AND FORMULATION

| CTFA Name | INCI | % | Supplier |
|---|---|---|---|
| Water | Aqua | 27.5-31.5 | Ten Li Distilled |
| | | | (local factory) |
| Propylene Glycol | Propylene Glycol | 26-30.5 | Lyondell Asia |
| | | | Pacific Ltd. |
| Sodium Stearate | Sodium Stearate | 12-13 | Stabilchem(M) |
| | | | Sdn Bhd |
| Isobutylene/MA | Isobutylene/MA | 4 | Rohm & Haas |
| Copolymer | Copolymer | | (TN Orotan) |
| Polyquaternium- | Polyquaternium- | 2 | International |
| 28 | 28 | | Supply Products |
| | | | (TN Gavquat) |
| Methylparaben | Methylparaban | 0.03 | Nipa Laboratories |
| | | | Ltd. |
| Sodium | Sodium | 0.06 | Taisho Technos |
| Dehydroacetate | Dehydroacetate | | Co., Ltd. |
| May Contain: | | to 100 | |
| (Pigment) | | | |
| Mica & Titanium | CI 77019 & | | Engelhard |
| Dioxide | CI 77891 | | Corporation/Mearl |
| Titanium Dioxide | CI 77891 | | Sun Chemical |
| D&C Red No. 27 | CI 45410 | | Sun Chemical |
| Al. Lake | | | |
| FD&C Blue | CI 42090 | | Sun Chemical |
| No. 1 Al. Lake | | | or Wackherr |
| Manganese Violet | CI 77742 | | Wackherr |
| D&C Red No. 7 | CI 15850 | | Wackherr |
| Cal. Lake | | | |
| Iron Oxides | CI 77491, CI77492, | | Wackherr |
| | CI77499 | | |
| FD&C Yellow | CI 19140 | | Wackherr |
| No. 5 Al. Lake | | | |

Notes:-
1. TN = Trade Name
2. % Variations due to different PH balance on different colours.

### Example 15

An example of a pink composition made according to gellan gum aspects of the present invention. In a mixing vessel, the following components were added:

| | | |
|---|---|---|
| Water | 56.6 | wt % |
| Thorcowet G-26 | 6 | wt % |
| Chemburst A-20FG | 0.5 | wt % |
| Isobutylene/Methyl Acrylate | 6 | wt % |
| FD&C Red #7 | 10 | wt % |
| Sodium Chloride | 0.75 | wt % |
| Titanium Dioxide | 3.6 | wt % |

This mixture is homogenized until the pigment is smooth and dispersed. The following components are then added:

| | | |
|---|---|---|
| Methyl Paraben | 0.03 | wt % |
| sodium dehydroacetate | 0.06 | wt % |
| Kelcogel F | 1 | wt % |
| Chemburst A-20FG | 0.5 | wt % |

The mixture is then heated to 85° C. and filled into cartridges.

Although the illustrative embodiments of the present disclosure have been described herein with reference to the accompanying drawings and examples, it is to be understood that the disclosure is not limited to those precise embodiments, and various other changes and modifications may be affected therein by one skilled in the art without departing from the scope of spirit of the disclosure. All such changes and modifications are intended to be included within the scope of the disclosure as defined by the appended claims.

1. A method for temporarily modifying the color of hair, comprising the steps of:
providing a composition comprising water, a co-solvent, a gelling agent selected from a soap gelling agent and a gellan gum gelling agent, and a coloring agent; and
applying the composition to hair;
wherein the composition removably adheres to the hair, and washes off with the application of a cleaning agent to the hair.

2. The method of claim 1, wherein the co-solvent is a glycol, and the gelling agent is a soap gelling agent.

3. The method of claim 2, wherein the glycol is propylene glycol and the soap gelling agent is sodium stearate.

4. The method of claim 1, wherein the composition comprises
from about 20 to about 35% by weight of water;
from about 20 to about 35% by weight of a co-solvent;
from about 10 to about 30% by weight of a soap gelling agent; and
from about 15 to about 30% by weight of a coloring agent.

5. The method of claim 4, wherein the composition further comprises from about 1 to about 5% by weight of a silicone polymer.

6. The method of claim 4, wherein the composition comprises:
from about 27 to about 32% by weight of water;
from about 26 to about 31% by weight of the co-solvent; and
from about 12 to about 15% by weight of soap gelling agent.

7. The method of claim 6, wherein the co-solvent is a glycol, and the soap gelling agent is a stearate salt.

8. The method of claim 7 wherein the glycol is propylene glycol, and the stearate salt is sodium stearate.

9. The method of claim 1 wherein the co-solvent is a glycol, and the gelling agent is a gellan gum gelling agent.

10. The method of claim 9, wherein the gellan gum gelling agent has low levels of acyl content.

11. The method of claim 10, wherein the composition comprises
from about 20 to about 65% by weight of water;
from about 10 to about 35% by weight of a co-solvent;
from about 0.1 to about 2% by weight of a low level acyl content gellan gum.

12. The method of claim 11, wherein the composition comprises:
from about 50 to about 65% by weight of water;
from about 10 to about 20% by weight of a co-solvent;
from about 0.5 to about 1.5% by weight of a low level acyl content gellan gum.

13. A temporary hair color composition comprising:
water;
a co-solvent;
a gelling agent selected from a soap gelling agent and a gellan gum gelling agent; and
a coloring agent,
wherein the temporary hair color composition is in a form suitable for application to hair.

14. The temporary hair color composition of claim 13, wherein the co-solvent is a glycol, and the gelling agent is a soap gelling agent.

15. The temporary hair color composition of claim 14, wherein the glycol is propylene glycol and the soap gelling agent is sodium stearate.

16. The temporary hair color composition of claim 13, wherein the composition comprises
from about 20 to about 35% by weight of water;
from about 20 to about 35% by weight of a co-solvent;
from about 10 to about 30% by weight of a soap gelling agent; and
from about 15 to about 30% by weight of a coloring agent.

17. The temporary hair color composition of claim 16, wherein the composition further comprises from about 1 to about 5% by weight of a silicone polymer.

18. The temporary hair color composition of claim 17, wherein the composition comprises:
from about 27 to about 32% by weight of water;
from about 26 to about 31% by weight of the co-solvent; and
from about 12 to about 15% by weight of soap gelling agent.

19. The temporary hair color composition of claim 18, wherein the co-solvent is a glycol, and the soap gelling agent is a stearate salt.

20. The temporary hair color composition of claim 19, wherein the glycol is propylene glycol, and the stearate salt is sodium stearate.

21. The temporary hair color composition of claim 13, wherein the gellan gum gelling agent has low levels of acyl content.

22. The temporary hair color composition of claim 21, wherein the composition comprises
from about 20 to about 65% by weight of water;
from about 10 to about 35% by weight of a co-solvent;
from about 0.1 to about 2% by weight of a low level acyl content gellan gum.

23. The temporary hair color composition of claim 22, wherein the composition comprises:
from about 50 to about 65% by weight of water;
from about 10 to about 20% by weight of a co-solvent;
from about 0.5 to about 1.5% by weight of a low level acyl content gellan gum.

24. A kit for temporarily modifying the color of hair, the kit comprising:
a package;
a coloring composition provided in the package, the coloring composition comprising water, a co-solvent, a gelling agent selected from a soap gelling agent and a gellan gum gelling agent, and a coloring agent; and
instructions provided on or in the package instructing a user to removably adhere the coloring composition to the user's hair by applying the coloring composition to the user's hair.

25. The kit of claim 24, wherein the instructions further include instructions for a user to remove the coloring composition by applying a cleaning agent to the hair.

26. The kit composition of claim 24, wherein the co-solvent is a glycol, and the gelling agent is a soap gelling agent.

27. The kit of claim 24, wherein the glycol is propylene glycol and the soap gelling agent is sodium stearate.

28. The kit of claim 24, wherein the composition comprises
from about 20 to about 35% by weight of water;
from about 20 to about 35% by weight of a co-solvent;
from about 10 to about 30% by weight of a soap gelling agent; and
from about 15 to about 30% by weight of a coloring agent.

29. The kit of claim 28, wherein the composition further comprises from about 1 to about 5% by weight of a silicone polymer.

30. The kit of claim 28, wherein the composition comprises:
from about 27 to about 32% by weight of water;
from about 26 to about 31% by weight of the co-solvent; and
from about 12 to about 15% by weight of soap gelling agent.

31. The kit of claim 30, wherein the co-solvent is a glycol, and the soap gelling agent is a stearate salt.

32. The kit of claim 31, wherein the glycol is propylene glycol, and the stearate salt is sodium stearate.

33. The kit of claim 24, wherein the gellan gum gelling agent has low levels of acyl content.

34. The kit of claim 33, wherein the composition comprises
from about 20 to about 65% by weight of water;
from about 10 to about 35% by weight of a co-solvent;
from about 0.1 to about 2% by weight of a low level acyl content gellan gum.

35. The temporary hair color composition of claim 34, wherein the composition comprises:
from about 50 to about 65% by weight of water;
from about 10 to about 20% by weight of a co-solvent;
from about 0.5 to about 1.5% by weight of a low level acyl content gellan gum.
